(19) United States
(12) Patent Application Publication
(10) Pub. No.: US 2015/0148680 A1
(43) Pub. Date: May 28, 2015

(54) ULTRASOUND PROBE

(71) Applicant: KONICA MINOLTA, INC., Chiyoda-ku, Tokyo (JP)

(72) Inventors: Michiyo Hirayama, Kanagawa (JP); Hideo Hongou, Kanagawa (JP)

(73) Assignee: KONICA MINOLTA, INC., Chiyoda-ku, Tokyo (JP)

(21) Appl. No.: 14/405,741

(22) PCT Filed: Jun. 12, 2013
(86) PCT No.: PCT/JP2013/003692

§ 371 Date: Dec. 4, 2014

(30) Foreign Application Priority Data
Jun. 14, 2012 (JP) 2012-135152

(51) Int. Cl. A61B 8/00

(52) U.S. Cl. CPC A61B 8/4444
USPC 600/459

(57) ABSTRACT

The disclosed technology relates to ultrasound probes, and the purpose of the technology is to prevent a housing and housing members from cracking and to ensure safety. According to this technology, in an ultrasound probe (12) accommodating one or more elements that include at least a piezoelectric element (3) inside a housing (10), the housing is formed of a plurality of housing members mutually joined. Inside the housing, protrusions having portions extending in a direction other than the direction perpendicular to the joined planes of the housing members are provided, and the housing is filled with a mold member (7) so as to cover the protrusions and the elements accommodated in the housing. The protrusions can be made together with the housing members as units, or can be fixed to the housing members. In another form, the protrusions can be fixed to an element accommodated in the housing.

### TECHNICAL FIELD

The present disclosure relates to an ultrasound probe used for observing, confirming and diagnosing the internal organs from the body surface or from the inside of the body cavity.

### BACKGROUND ART

Conventional ultrasound probes include a housing (which is also called “casing,” “housing case” or the like) that houses therein a piezoelectric element section, an electric signal line and the like, and in order to fix an electric signal line and the like in the housing and to improve electrical isolation between signal lines, the inside of the housing is filled with a synthetic resin mold (see, for example, PTL 1).

In addition, the housing is composed of a combination of a plurality of housing members, and the inside of the housing is filled with the synthetic resin mold after the electric signal line and the like are housed in the housing and the housing members are bonded together with an adhesive agent (see, for example, PTL 2).

### CITATION LIST

### Patent Literature

### PTL 1

### Japanese Patent Application Laid-Open No. 10-85219 (ABSTRACT and FIG. 1)

### PTL 2

Japanese Patent Application Laid-Open No. 11-318897 (paragraphs [0024] to [0028] and FIG. 1)

### SUMMARY OF INVENTION

### Technical Problem

However, in the conventional housing composed of a plurality of housing members, the thermal expansion and the thermal contraction are different due to the difference between the synthetic resin material of the molding material and the housing itself, and consequently the housing members may be separated at the coupling surfaces. Further, the housing may crack due to excessive load stress of the external force exerted by bending and twisting when it is wiped for cleaning, for example.

An object of the present disclosure is to solve the problems of the conventional art, and according to an aspect of the present disclosure, a safe ultrasound probe which can prevent crack of a housing is provided.

An ultrasound probe according to an aspect of the present disclosure is an ultrasound probe in which one or more components including at least a piezoelectric element section are housed in a housing, in which: the housing is composed of a plurality of housing members coupled to each other; a protrusion including a portion extending in a direction other than a direction perpendicular to coupling surfaces of the housing members is provided in the housing; and an inside of the housing is filled with a molding material so as to surround the protrusion and the one or more components housed in the housing.

Preferably, the protrusion includes, in addition to the portion extending in the direction other than the direction perpendicular to the coupling surface, a portion extending in a direction perpendicular to the coupling surface.

Preferably, the ultrasound probe further comprises a cable housed in the housing, in which, relative to an intermediate point of the housing member between a distal end portion at which ultrasound waves are transmitted and received and an end portion on a side opposite to the distal end portion in an axial direction of the cable, the protrusion is located at a position on the distal end portion side.

Preferably, the protrusion is molded integrally with the housing member, or fixed to the housing member.

Preferably, the protrusion is molded integrally with at least one of the components housed in the housing, or fixed to at least one of the components.

Preferably, the protrusion includes a member formed of a metal.

Preferably, the molding material filling the housing is an epoxy resin.

Preferably, the molding material filling the housing is a urethane resin.

Preferably, the molding material filling the housing has a two-layer structure of an epoxy resin and a urethane resin.

According to an aspect of the present disclosure, since the above-mentioned configuration is employed, it is possible to prevent the difference in thermal expansion or thermal contraction due to the difference between the synthetic resin material of the molding material and housing members of a housing, and to prevent separation of the housing members due to excessive load stress of the external force exerted by bending and twisting, and thus, safety is improved.

### BRIEF DESCRIPTION OF DRAWINGS

FIG. 1 is a perspective view illustrating an ultrasound probe according to Embodiment 1 of the present disclosure;

FIG. 2 is an exploded perspective view illustrating the ultrasound probe according to Embodiment 1 of the present disclosure;

FIG. 3 is a longitudinal sectional view illustrating the ultrasound probe according to Embodiment 1 of the present disclosure;

FIG. 4 is a front view illustrating a housing member of the ultrasound probe according to Embodiment 1 of the present disclosure;

FIG. 5 is a sectional view taken along line A-A of FIG. 3;

FIG. 6 is a sectional view taken along line B-B of FIG. 3;

FIG. 7 is an exploded perspective view illustrating an ultrasound probe according to Embodiment 2 of the present disclosure;

FIG. 8 is a front view illustrating a housing member of the ultrasound probe according to Embodiment 2 of the present disclosure;

FIG. 9 is a longitudinal sectional view illustrating the ultrasound probe according to Embodiment 2 of the present disclosure;

FIG. 10 is a sectional view taken along line A-A of FIG. 9; and

FIG. 11 is a sectional view taken along line B-B of FIG. 9.

### DESCRIPTION OF EMBODIMENTS

In the following, an ultrasound probe according to embodiments of the present disclosure (also referred to as “ultrasound probe,” or simply referred to as “probe”) will be described with reference to the accompanying drawings.

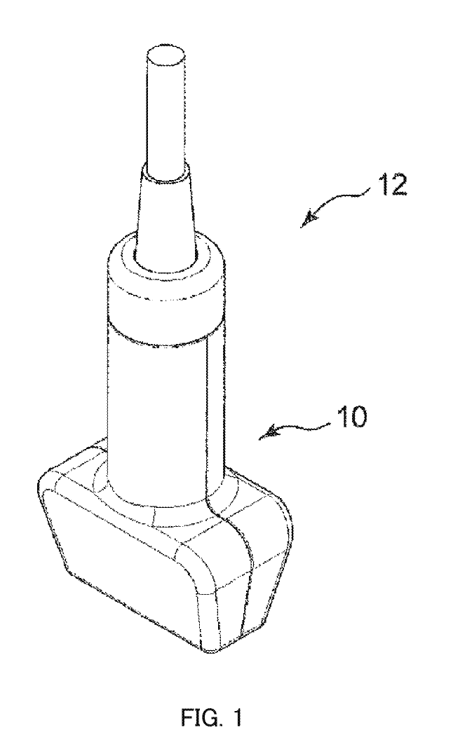

FIG. 1 is a perspective view illustrating an ultrasound probe according to Embodiment 1 and Embodiment 2 of the present disclosure, and illustrates an exemplary external appearance of ultrasound probe 12. It is to be noted that the present disclosure is not limited to the external appearance of a linear scanning type ultrasound probe illustrated in FIG. 1, and may be applied to convex scanning type ultrasound probes and electronic sector scanning type ultrasound probes. Further, the present disclosure may be applied to ultrasound probes intended for the diagnosis from the inside of the body cavity, as well as ultrasound probes intended for the diagnosis from the body surface. As illustrated in FIG. 1, the main part of ultrasound probe 12 is composed of housing 10 and components housed in housing 10.

### Embodiment 1

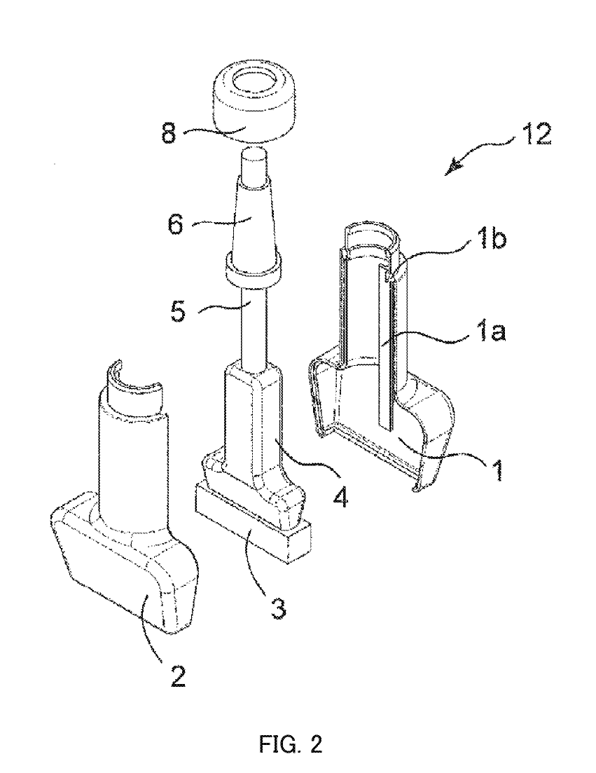

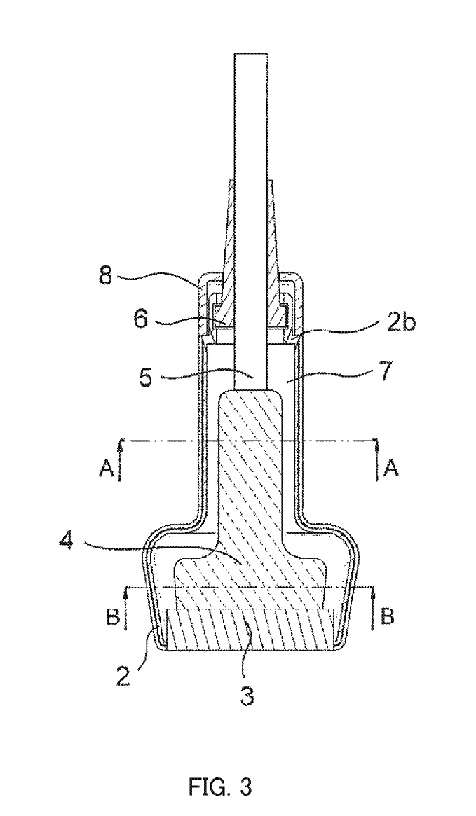

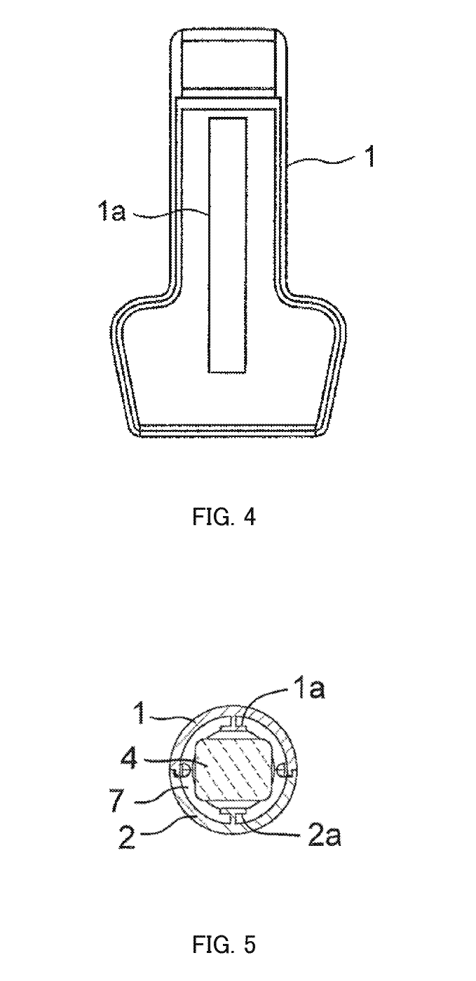

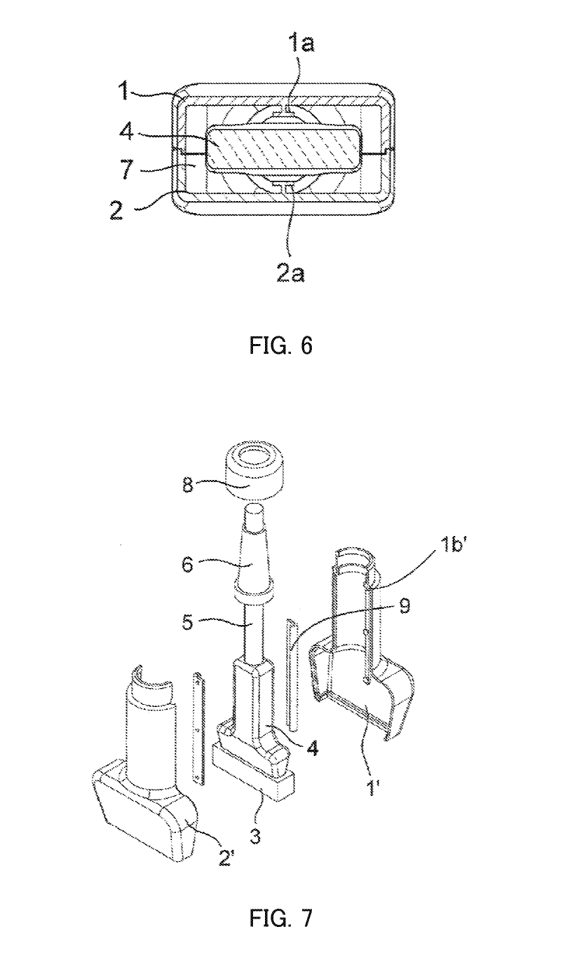

FIG. 2 is an exploded perspective view illustrating the ultrasound probe according to Embodiment 1 of the present disclosure. The ultrasound probe mainly includes piezoelectric element section 3 that transmits and receives ultrasound waves, cable 5 that transmits an electric signal to an ultrasound diagnostic apparatus (not illustrated), electric signal line connection section 4 that connects piezoelectric element section 3 and cable 5 together, and housing 10 that covers these components and serves as a holding section for the operator (see FIG. 1). It is to be noted that housing 10 may be composed of a plurality of housing members, and in the example illustrated in FIG. 2, housing 10 is composed of a combination of two housing half bodies of housing member 1 and other housing member 2. FIG. 3 is a longitudinal sectional view illustrating Embodiment 1. FIG. 5 and FIG. 6 are sectional views taken along line A-A and line B-B of FIG. 3, respectively, as viewed in the arrow direction. FIG. 4 is a front view illustrating housing member 1.

After internal components such as piezoelectric element section 3, electric signal line connection section 4, cable 5, and cable bushing 6 that prevents disconnection due to abrupt bending of cable 5 or the like are coupled together, housing member 1 and housing member 2 are coupled together and bonded together with an adhesive agent (such as silicone adhesive agent) so as to cover the internal components. Thus, housing member 1 and housing member 2 are integrated. After housing member 1 and housing member 2 are integrated, heat-curable or naturally-curable liquid of synthetic resin molding material 7 is injected from synthetic resin mold filling port 1b (2b, in FIG. 3) in order to fix the internal components, to improve the electrical isolation for preventing short circuit of an electric signal, and to prevent separation of solder connections and connector connections such as the electrical connection between a piezoelectric element section and an electric signal line. After the injection, synthetic resin molding material 7 is cured in the housing, and synthetic resin mold filling port 1b is covered with cap 8 so that the port cannot be seen from the outside.

The ultrasound probe according to Embodiment 1 of the present disclosure is described in detail with reference to FIG. 3, FIG. 5 and FIG. 5. As illustrated in the sectional views of FIG. 5 and FIG. 6, housing member 1 and housing member 2 are provided with protrusion la and protrusion 2a each having a portion extending in a direction other than a direction (horizontal direction in FIG. 3) perpendicular to the coupling surfaces of the housing members (the surfaces including end portions to be coupled in contact with each other; in FIG. 5, the surfaces which are disposed between housing members 1 and 2, include line laterally extending at a substantially center portion in the drawing, and are perpendicular to the paper surface). Synthetic resin molding material 7 flows in the housing in such a manner as to surround the above-mentioned extending portions of protrusions 1a and 2a, and thereafter synthetic resin molding material 7 is cured. That is, since the above-mentioned extending portions of protrusions 1a and 2a extend in a direction different from the direction in which housing member 1 and housing member 2 are separated (direction perpendicular to the coupling surfaces thereof), the attraction and mutual fixation between housing member 1 and housing member 2 are strengthened, and it is possible to prevent the housing members from separating in the direction perpendicular to the plane of the coupling surface due to the difference in thermal expansion, thermal contraction or the like of the synthetic resin material, or the external force exerted by bending and twisting.

In FIG. 5, the protrusion has a so-called T-shape composed of a first portion extending from the housing member toward the inside of the housing in a direction perpendicular to the coupling surfaces of the housing members, and a second portion that extends in a horizontal direction with respect to the coupling surface and is joined at a center portion thereof with an end portion of the first portion different from the junction between the housing member and the first portion; however, the shape of the protrusion is not limited to the T-shape. It is only necessary that a portion extending in a direction other than a direction perpendicular to the coupling surfaces of the housing members is provided at a position remote from the housing member. It is also possible to employ a configuration having a first portion extending from the housing member toward the inside of the housing in the direction perpendicular to the coupling surfaces of the housing members, and a second portion extending from a position of the first portion remote from the junction between the housing member and the first portion in a direction other than a direction perpendicular to the coupling surface. In addition, as described later in Embodiment 2, a so-called J-shape or a so-called H-shape may be employed. The H-shape includes a third portion extending in a direction perpendicular to the coupling surfaces of the housing members, at the both ends of the second portion of the above-described T-shape. It is also possible to employ a configuration in which an H-shape including a first portion horizontally extending with respect to the coupling surfaces of the housing members, a second portion extending from a substantially center of the first portion in a direction perpendicular to the coupling surface, a third portion horizontally extending with respect to the coupling surface at an end portion of in the second portion different from the end portion at which the first portion is connected, is joined to the housing member at the first portion, for example. The H-shape and the housing member may be joined with each other with one or more members extending in the direction perpendicular to the first portion.

It is to be noted that protrusion 1a and protrusion 2a may be molded integrally with housing member 1 and housing member 2, or may be provided as separate components and fixed to housing member 1 and housing 2 by as in Embodiment 2 described later.

### Embodiment 2

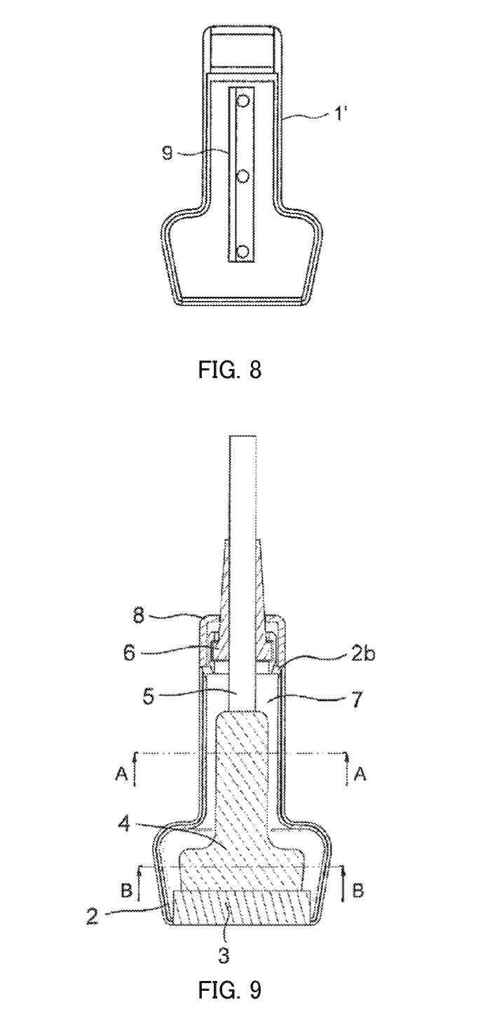

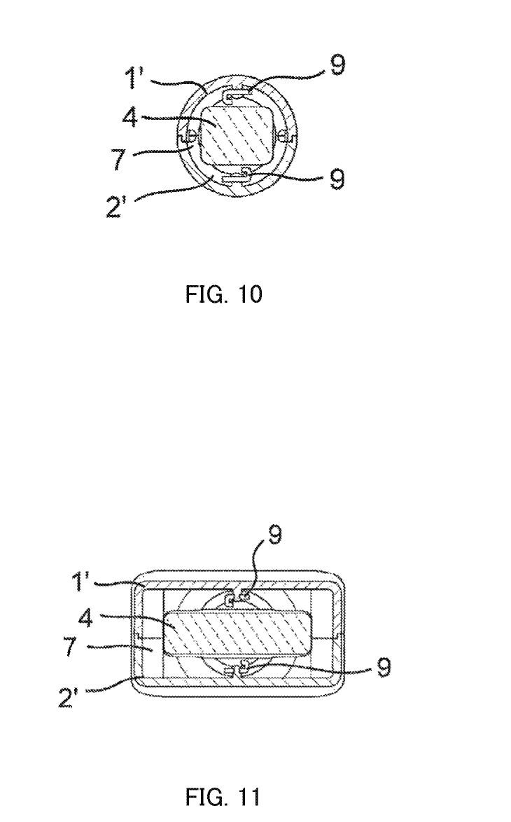

FIG. 7 is an exploded perspective view illustrating an ultrasound probe according to Embodiment 2 of the present disclosure. In Embodiment 2, same components as those in Embodiment 1 are denoted by the same reference symbols, and the components which are different from those in Embodiment 1 but correspond to those in Embodiment 1 are denoted by the same reference symbols or by the same reference symbols attached with “′.” In addition, FIG. 8 is a front view illustrating housing member 1′ in Embodiment 2. In housing member 1′, plate 9 extends from a portion in the proximity of an end portion on the cap side toward the probe end side in the cable axial direction, up to a position nearer to the probe end side of the housing member relative to the intermediate point between the probe end side and the other end portion opposite to the probe end side. In addition, plate 9 is fastened with screws at its both end portions and center portion. FIG. 9 is a longitudinal sectional view of Embodiment 2, and FIG. 10 and FIG. 11 are respectively sectional views taken along line A-A and line B-B in FIG. 9 as viewed in the arrow direction. Embodiment 2 is different from Embodiment 1 in that the protrusion is provided as a separate component, which is provided integrally with housing member 1 or housing member 2 at the inner wall of the housing members in Embodiment 1 as described above. In Embodiment 2, for the purpose of simplifying the shape of the metal mold, the protrusion is provided by fixing plate 9 on a rib provided on the inner wall of housing member 1 or housing member 2 by welding. The other configurations in Embodiment 2 are same as those of Embodiment 1. It is to be noted that the fixation of plate 9 to the rib provided on the inner wall of housing member 1 or housing member 2 may be achieved by bonding or screwing, or both. Since protrusions that cause undercut are not formed in housing member 1 and housing member 2 in this configuration, the metal mold can be simplified.

In addition, plate 9 may be formed by a metal (for example, stainless steel), and in that case, in comparison with the case where plate is formed only of synthetic resins, the rigidity is increased, and the tolerance against the stress exerted by the external force is further improved.

Further, when plate 9 is not a flat plate but has a J-shape as viewed in cross section in a longitudinal direction of an ultrasound probe as in FIG. 10, the rigidity against bending and twisting is further increased. Here, the J-shape has a shape that has first to third portions and is connected to the housing member at the first portion thereof, for example. Specifically, in the J-shape, the first portion horizontally extending with respect to the coupling surface of the housing member, and a second portion vertically extending with respect to the coupling surface from one end portion of the first portion are connected together, and a third portion having a length shorter than that of the first portion and horizontally extending with respect to the coupling surface is connected to the second portion at an end portion of the second portion different from an end portion of the second portion at which the first portion is connected. It is to be noted that the second portion has a curved shape and is smoothly joined with the first portion and the third portion. The J-shape and the housing member may be joined together with a member extending in a direction perpendicular to the first portion. In addition, the cross-sectional shape may not be the J-shape, and may be an H-shape or a T-shape as described in Embodiment 1 as long as a portion extending in a direction other than a direction perpendicular to the coupling surfaces of the housing members is provided at a position remote from the housing member. It is also possible to employ a configuration having a first portion extending from the housing member toward the inside of the housing in the direction perpendicular to the coupling surfaces of the housing members, and a second portion extending from a position of the first portion remote from the junction between the housing member and the first portion in a direction other than a direction perpendicular to the coupling surface. While the H-shape and the T-shape are not greatly differ from each other in terms of the effect of improvement in rigidity, the space for welding housing member 1 and housing member 2 and favorable workability are ensured when the J-shape is employed, and, when the direction of the J-shape of plate 9 of welding fixation to housing member 1 and housing member 2 is inverted, the fixation between housing member 1 and housing member 2 is strengthened, and the mass balance is maintained. Preferably, plates 9 joined to housing member 1 and housing member 2 have the same shape and are joined to housing member 1 and housing member 2 in a point-symmetrical manner.

Additionally, when metal plate 9 is used, electrical effect caused by the contact with the GND process sections such as electric signal line connection section 4 can be prevented by applying an insulation process. Examples of the method of the insulation process include synthetic resin coating, baking painting, and chromium plating.

Examples of synthetic resin molding material 7 in Embodiment 1 and Embodiment 2 include epoxy resin and foaming urethane resin. It is also possible to employ a two-layer structure by combining the above-mentioned materials.

In the above-mentioned Embodiment 1 and Embodiment 2, the housing is composed of two housing members; however, the number of the housing members of the housing is not limited to two, and any number of the housing members may be employed to compose the housing. In addition, the housing members may not be the housing members which are divided in a radial direction (lateral direction) with the longitudinal direction or the cable axial direction as the center as in Embodiment 1 and Embodiment 2, and, may be a housing members that are divided in the longitudinal direction or in the cable axial direction (vertical direction), for example.

In either of Embodiment 1 or Embodiment 2, preferably, the protrusion is provided at least at a position at which electric signal line connection section 4 is provided, in the cable axial direction.

In addition, regarding the problem of separation of the housing members, the closer to the probe end position remote from cap 8 where the connection between the housing members is not reinforced by cap 8, the more the separation occurs. Therefore, it is more advantageous to provide the protrusion described in Embodiment 1 and Embodiment 2 on a side closer to the probe end side relative to an intermediate point between the probe end side of the housing member and the end portion on side opposite to the probe end side, that is, an intermediate point in the longitudinal direction of the ultrasound probe, in the cable axial direction. It is to be noted that, as described in Embodiment 1 and Embodiment 2, the shape of the protrusion may not be a linear shape. It is also possible to intermittently provide a plurality of the protrusions in the cable axial direction, and to provide the protrusions in a plurality of lines.

In addition, the protrusion is molded integrally with housing members 1 and 2 in Embodiment 1, and the protrusion is fixed to housing members 1 and 2 in Embodiment 2; however, the present disclosure is not limited to this. The protrusion may be provided integrally with one or more of the components provided in the housing such as the electric signal line connection section and the cable section, or may be fixed to one or more of the components. Also with such a configuration, the effect of preventing the separation of the housing members can be obtained. However, providing the protrusion to the housing member itself can prevent the separation of the housing members more surely in comparison with the other configurations, and therefore a proper configuration can be selected as necessary.

### INDUSTRIAL APPLICABILITY

As has been described so far, by employing the above-mentioned configuration, the ultrasound probe according to the present disclosure can advantageously prevent the difference in thermal expansion or thermal contraction due to the difference between the synthetic resin material of the molding material and the housing, and crack of the housing due to excessive load stress of the external force exerted by bending and twisting, and can be adopted to ensure the safety of the ultrasound probe. Thus, the ultrasound probe according to the present disclosure is widely applicable in the industry relating to designing or manufacturing the ultrasound probe, and the medical industry, the examination industry, and industries in which various comparative fracture tests of products are conducted in which the ultrasound probe is utilized.

1. An ultrasound probe in which one or more components including at least a piezoelectric element section are housed in a housing, wherein:
the housing is composed of a plurality of housing members coupled to each other;
a protrusion including a portion extending in a direction other than a direction perpendicular to coupling surfaces of the housing members is provided in the housing; and
an inside of the housing is filled with a molding material so as to surround the protrusion and the one or more components housed in the housing.

2. The ultrasound probe according to claim 1, wherein the protrusion includes, in addition to the portion extending in the direction other than the direction perpendicular to the coupling surface, a portion extending in a direction perpendicular to the coupling surface.

3. The ultrasound probe according to claim 1 further comprising a cable housed in the housing, wherein, relative to an intermediate point of the housing member between a distal end portion at which ultrasound waves are transmitted and received and an end portion on a side opposite to the distal end portion in an axial direction of the cable, the protrusion is located at a position on the distal end portion side.

4. The ultrasound probe according to claim 1, wherein the protrusion is molded integrally with the housing member, or fixed to the housing member.

5. The ultrasound probe according to claim 1, wherein the protrusion is molded integrally with at least one of the components housed in the housing, or fixed to at least one of the components.

6. The ultrasound probe according to claim 1, wherein the protrusion includes a member formed of a metal.

7. The ultrasound probe according to claim 1, wherein the molding material filling the housing is an epoxy resin.

8. The ultrasound probe according to claim 1, wherein the molding material filling the housing is a urethane resin.

9. The ultrasound probe according to claim 1, wherein the molding material filling the housing has a two-layer structure of an epoxy resin and a urethane resin.
